SEQUENCE LISTING (12 sequences)

SEQ ID NO: 1 - protein, 409 aa, Arabidopsis thaliana
MAFEKIKVANPIVEMDGDEMTRVIWKSIKDKLITPFVELDIKYFDLGLPHRDATDDKVTIESAEATKKYNVAIKCATITPDEGRVTEFGLKQMWRSPNGTIRNILNGTVFREPIICKNVPKLVPGWTKPICIGRHAFGDQYRATDAVIKGPGKLTMTFEGKDGKTETEVFTFTGEGGVAMAMYNTDESIRAFADASMNTAYEKKWPLYLSTKNTILKKYDGRFKDIFQEVYEASWKSKYDAAGIWYEHRLIDDMVAYALKSEGGYVWACKNYDGDVQSDFLAQGFGSLGLMTSVLVCPDGKTIEAEAAHGTVTRHFRVHQKGGETSTNSIASIFAWTRGLAHRAKLDDNAKLLDFTEKLEAACVGTVESGKMTKDLALIIHGSKLSRDTYLNTEEFIDAVAAELKERLN

SEQ ID NO: 2 - protein, 411 aa, Fragaria vesca
MAFEKIKVANPIVEMDGDEMTRIFWQSIKDKLILPFLDLDIKYFDLGLTHRDATDDKVTIESAEATLKYNVAIKCATITPDEARMKEFNLKQMWRSPNGTIRNILNGTVFREPILCKNIPRLIPGWTKPICIGRHAFGDQYRATDTVIKGPGKLKLMFVPEGQDEKMEFEVFNFTGEGGVAIAMYNTDESIRAFAEASMNTAYEKKWPLYLSTKNTILKKYDGRFKDIFQEVYEAHWKSKYEAAGIWYEHRLIDDMVAYALKSEGGYVWACKNYDGDVQSDFLAQGFGSLGLMTSVLVCPDGKTIEAEAAHGTVTRHYRVHQRGGETSTNSIASIFAWTRGLAHRAKLDDNAKLLEFTQKLEEACIGTVESGKMTKDLALILHGPKLARNHYLNTEEFIDAVAAELKAKLC

SEQ ID NO: 3 - protein, 412 aa, Malus domestica
MAFQKIKVANPIVEMDGDEMTRVFWKSIKDKLILPFVELDIKYFDLGLPHRDATDDKVTVESAEATLKYNVAIKCATITPDEARMKEFSLKSMWRSPNGTIRNILNGTVFREPILCKNIPRLIPGWTKPICIGRHAFGDQYRATDAVIKGPGKLKLVFVPEGKDEKTELDVYDFTGEGGVALAMYNTDESIRAFAEASMTTAYEKKWPLYLSTKNTILKKYDGRFKDIFQEVYEANWKSKFDAAGIWYEHRLIDDMVAYALKSDGAYVWACKNYDGDVQSDMLAQGFGSLGLMTSVLVCPDGKTIEAEAAHGTVTRHYRVHQKGGETSTNSIASIFAWTRGLAHRAKLDDNARLLEFTQKLEEACIGTVESGKMTKDLALILHGSKLARNHYLNTEEFIDAVAEELKAKLAC

SEQ ID NO: 4 - protein, 415 aa, Nicotiana tabacum
MAFDKIKVENPIVEMDGDEMTRVIWKSIKDKLICPFLELDIKYFDLGLPHRDATDDKVTVESAEATQKYNVAIKCATITPDEARVKEFNLKSMWRSPNGTIRNILNGTVFREPIMCKNIPRLVPGWTKPICIGRHAFGDQYRATDTVIQGAGKLKLVFVPEGTDEKTEFEVYNFTGAGGVALSMYNTDESIRSFAEASMNMAYQKKWPLYLSTKNTILKKYDGRFKDIFQEVYEANWKSKYEEAGIWYEHRLIDDMVAYALKSEGGYVWACKNYDGDVQSDFLAQGFGSLGLMTSVLVCPDGKTIEAEAAHGTVTRHYRVHQKGGETSTNSIASIFAWTRGLAHRATLDNNERLLDFTEKLEAACIGAVESGKMTKDLALIIHGSKLSRDHYLNTEEFIDAVADELKARLLKAKA

SEQ ID NO: 5 - protein, 415 aa, Solanum annum
MAFQKINVQNPIVEMDGDEMTRVIWKSIKDKLILPFLELDIKYFDLGLPHRDLTDDKVTVESAEATLKYNVAIKCATITPDEARVKEFKLKSMWRSPNGTIRNILNGTVFREPIMCKNIPRLVPGWTKPICIGRHAFGDQYRATDAVIQGAGKLKLVFVPEGSDEKTEYEVYNFTGAGGVALSMYNTDESIRAFADASMNMAYQKKWPLYLSTKNTILKKYDGRFKDIFQEVYEASWKSKYEEAGIWYEHRLIDDMVAYALKSEGGYVWACKNYDGDVQSDFLAQGFGSLGLMTSVLVCPDGKTIEAEAAHGTVTRHYRVHQKGGETSTNSIASIFAWTRGLAHRATLDKNERLLDFTEKLEAACIGAVESGKMTKDLALIIHGSKLSREHYLNTEEFIDAVADELKAKLLKAKA

SEQ ID NO: 6 - protein, 415 aa, Solanum melongena
MAFQKIVVQNPIVEMDGDEMTRVIWKSIKDKLILPFLELDIKYFDLGLPHRDATDDKVTVESAEATLKYNVAIKCATITPDEARVKEFNLKSMWRSPNGTIRNILNGTVFREPIMCKNIPRLVPGWTKPICIGRHAFGDQYRATDTVIKGAGKLKLVFVPEGSGEKSELEVYNFTGAGGVALSMYNTDESIRSFAEASMNMAFQKKWPLYLSTKNTILKKYDGRFKDIFQEVYEANWKSKYEEAGIWYEHRLIDDMVAYALKSEGGYVWACKNYDGDVQSDFLAQGFGSLGLMTSVLVCPDGKTIEAEAAHGTVTRHYRVHQKGGETSTNSIASIFAWTRGLAHRATLDKNERLLDFTEKLEAACIGAVESGKMTKDLALIIHGSKLSREHYLNTEEFIDAVADELKAKLLKAKA

SEQ ID NO: 7 - protein, 415 aa, Solanum tuberosum
MAFQKITVQNPIVEMDGDEMTRVIWKSIKDKLILPFLELDIKYFDLGLPHRDATDDKVTVESAEATQKYNVAIKCATITPDEARVTEFNLKSMWRSPNGTIRNILNGTVFREPIMCKNIPRLVPGWTKPICIGRHAFGDQYRATDTVIKGAGKLKLVFVPEGSDEKTEFEVYNFTGAGGVALSMYNTDESVRSFAEASMNMAFQKKWPLYLSTKNTILKKYDGRFKDIFQEVYEANWKSKYEEAGIWYEHRLIDDMVAYALKSEGGYVWACKNYDGDVQSDFLAQGFGSLGLMTSVLVCPDGKTIEAEAAHGTVTRHYRVHQKGGETSTNSIASIFAWTRGLAHRATLDNNERLLDFTEKLEAACIGAVESGKMTKDLALIIHGSKLSREHYLNTEEFIDAVADELKARLLKAKA

SEQ ID NO: 8 - protein, 415 aa, Solanum lycopersicum
MAFQKIIVQNPIVEMDGDEMTRVIWKSIKDKLILPFLELDIKYFDLGLPHRDATDDKVTIESAEATQKYNVAIKCATITPDEARVKEFNLKSMWRSPNGTIRNILNGTVFREPIMCKNIPRLVPGWTKPICIGRHAFGDQYRATDTVIKGAGKLKLVFVPEGSDEKTEFEVYNFTGAGGVALSMYNTDESVRAFAEASMNMAFQKKWPLYLSTKNTILKKYDGRFKDIFQEVYEANWKSKYEEAGIWYEHRLIDDMVAYALKSEGGYVWACKNYDGDVQSDFLAQGFGSLGLMTSVLVCPDGKTIEAEAAHGTVTRHYRVHQKGGETSTNSIASIFAWTRGLAHRATLDNNERLLDFTEKLEAACIGAVESGKMTKDLALIIHGSKLSREHYLNTEEFIDAVADELKAKLLKAKA

SEQ ID NO: 9 - protein, 410 aa, Musa acuminata
MAFEKIKVFNPIVEMDGDEMTRVFWKSIKEKLIFPFLDLDIKYFDLGLPNRDATDDKVTIESAEATLNYNVAIKCATITPDEARVKEFNLKSMWKSPNGTIRNILNGTVFREPIICKNIPRLVPGWTKPICIGRHAFGDQYRATDTVIKGPGKLKLVFEGKDEEVELEVFNFTGAGGVALSMYNTDESIRAFADASMATAYQKKWPLYLSTKNTILKKYDGRFKDIFQEVYETEWKSKFEAAGIWYEHRLIDDMVAYALKSEGGYVWACKNYDGDVQSDFLAQGFGSLGLMTSVLVCPDGKTIEAEAAHGTVTRHYRVHQKGGETSTNSIASIFAWSRGLAHRAKLDDNARLLDFTEKLEAACVGTVESGKMTKDLALLIHGSSVTRAQYLNTEEFIDAVASELRARLSA

SEQ ID NO: 10 - DNA, 26 bp, Artificial Sequence
  Synthetic
ccauucuuaagaaauaugaugggagg

SEQ ID NO: 11 - DNA, 32 bp, Solanum lycopersicum
ataccattcttaagaaatatgatgggaggttc

SEQ ID NO: 12 - DNA, 33 bp, Artificial Sequence
  Synthetic
ataccattctttaagaaatatgatgggaggttc

(19) United States
(12) Patent Application Publication
(10) Pub. No.: US 2023/0135761 A1
(43) Pub. Date: May 4, 2023

(54) TRANSGENIC PLANTS HAVING PROLONGED RIPENING AND REDUCED SUSCEPTIBILITY TO PATHOGENS

(71) Applicant: MIGAL GALILEE RESEARCH INSTITUTE LTD, Kiryat Shmona (IL)

(72) Inventors: Dan GAMRASNI, Shamir (IL); Martin GOLDWAY, Timrat (IL)

(73) Assignee: MIGAL GALILEE RESEARCH INSTITUTE LTD, Kiryat Shmona (IL)

(21) Appl. No.: 17/911,794

(22) PCT Filed: Mar. 15, 2020
(86) PCT No.: PCT/IL2020/050307

§ 371 Date: Sep. 15, 2022

(51) Int. Cl. C12N 15/82; C12N 9/04; A23B 7/154

(52) U.S. Cl. CPC C12N 15/8249; C12N 9/0006; C12Y 101/01042; C12N 15/8213; C12N 15/8279; A23B 7/154

(57) ABSTRACT

The present invention is directed to a transgenic plant including a mutated form of a cytosolic isocitrate dehydrogenase (ICDH) encoding gene, wherein the transgenic plant has a reduced ethylene production rate, a reduced respiration rate or both. Further provided are methods for prolonging the development to ripening period of a plant or a part derived from same, and for producing an agent suitable for increasing shelf life of a plant or a part derived from same.

### FIELD OF THE INVENTION

The present invention relates in general to the field of plant physiology, and is directed to transgenic plants and methods for prolonging fruit ripening and shelf life and/or reducing susceptibility to pathogens.

### BACKGROUND

At the onset of ripening of climacteric fruit respiration increases usually in concert with a boost in the production of the hormone ethylene. In contrast, in non-climacteric fruit respiration does not increase as ripening proceeds and the fruit does not require ethylene for completion of fruit maturation. Economically important fruit such as avocado, banana, apple and tomato are climacteric whereas citrus, grape, strawberry and pepper are non-climacteric. Application of exogenous ethylene to mature climacteric fruit stimulates ethylene biosynthesis and respiration inducing rapid fruit ripening. In non-climacteric fruits this treatment also stimulates respiration, however, ethylene production is not induced, and the fruits do not ripen, and only marginal effects can be observed, for example the chlorophyll degradation in citrus fruits.

For commercial purposes, climacteric fruits are harvested upon completion of their growth period and their ripening continues thereafter with ethylene evolution playing a major role during storage and marketing.

Tomato serves as a model system for research of climacteric fruits since its ripening takes only several days that are accompanied by easily monitored changes, such as: conversion of color, reduction in firmness, increase in sugar content and/or production, decrease in starch content and/or production, and development of aroma.

Many of the genes involved in climacteric ripening were first identified in tomato. For example, the key ripening genes Ripening-Inhibitor (RIN), Tomato Agamos-Like1 (TAGL1) MADS-box, Squamosa Promotor Binding Protein (SBP), and Colorless Non Ripening (CNR), which are necessary for the progression of virtually all ripening processes and many more including signal transduction components that respond to the hormonal and environmental stimuli and modulate ripening phenotypes. Orthologs to the ripening tomato genes were detected in various other climacteric and non-climacteric species. Hence, it was apparent that despite significant differences in the morphological, physiological, and biochemical characteristics of fruit in different species, the ripening mechanisms are generally conserved.

The ethylene action inhibitor 1-methylcyclopropene (1-MCP) is applied commercially as a postharvest agent, dramatically prolonging storage and shelf life of many different fruit, flowers and leaves. 1-MCP, like ethylene, is a gaseous molecule that binds to the ethylene receptors and effectively inhibits ethylene action. Screens applying 1-MCP have been used to identify genes of the ethylene pathway and to define ethylene dependent ripening processes.

There is still a great need for agents targeting the molecular link between ethylene production and climacteric respiration so as to prolong ripeninig and shelf life periods and possibly reduce susceptability to pathogens.

### SUMMARY

According to a first aspect, there is provided a transgenic plant comprising at least one cell comprising at least one mutation in a cytosolic isocitrate dehydrogenase (ICDH) encoding gene, wherein the transgenic plant has a reduced ethylene production rate compared to control, a reduced respiration rate compared to control, or both.

According to another aspect, there is provided a plant part derived from the herein disclosed transgenic plant.

According to another aspect, there is provided a method comprising: contacting a plant or a part derived therefrom with an effective amount of an agent, wherein the agent is capable of reducing cytosolic ICDH activity in the plant or the plant part.

According to another aspect, there is provided a method for producing an agent suitable for increasing shelf life of a plant or a part derived therefrom, comprising: obtaining an agent that reduces cytosolic NADP-dependent ICDH activity, determining the ethylene production rate, respiration rate, or both in the presence of the obtained agent, and selecting at least one agent the reduces ethylene production rate, respiration rate, or both, thereby producing an agent suitable for increasing the shelf life of a plant or a part derived therefrom.

In some embodiments, the transgenic plant comprises at least one exogenous polynucleotide comprising the at least one mutation.

In some embodiments, the control is a genetic reference plant comprising the same genotype of the transgenic plant and is lacking the at least one mutation.

In some embodiments, the cytosolic ICDH comprising the mutation has a reduced NADP-dependent ICDH activity compared to a control ICDH, or is inactive.

In some embodiments, the at least one cytosolic ICDH encoding gene is selected from the group consisting of: ICDH1, ICDH2cyt, ICDH11, and any combination thereof.

In some embodiments, the at least one cytosolic ICDH encoding gene is ICDH1.

In some embodiments, the plant part is selected from the group consisting of: a fruit, a flower, a seed, and a leaf.

In some embodiments, the plant part is a fruit.

In some embodiments, the fruit is characterized by having a low climacteric peak on days 15-21 after harvest.

In some embodiments, the fruit has a development to ripening period at least 10% longer than a fruit derived from the genetic reference plant.

In some embodiments, the plant part has a shelf life increased by at least 7 days compared to a plant part derived from the genetic reference plant.

In some embodiments, the contacting results in: reduction of ethylene production rate, reduction of respiration rate, increase of shelf life, in the plant or the part derived therefrom, or any combination thereof.

In some embodiments, the ICDH activity is NADP-dependent activity.

In some embodiments, the ICDH is selected from the group consisting of: ICDH1, ICDH2cyt and ICDH11.

In some embodiments, reducing cytosolic NADP-dependent ICDH activity is constitutively or permanently reducing.

In some embodiments, the agent comprises at least one polynucleotide molecule.

In some embodiments, the at least one polynucleotide molecule encodes at least one component of the CRISPR-Cas system.

In some embodiments, the at least one component of the CRISPR-Cas system is a guide RNA (gRNA) or a Cas protein.

In some embodiments, the gRNA comprises the nucleic acid sequence 5′-CCATTCTTAAGAAATATGATGGGAGG-3′ (SEQ ID NO: 10).

In some embodiments, the Cas is Cas9.

In some embodiments, the contacting is: pre-harvest contacting, post-harvest contacting, or both.

In some embodiments, the contacting results in the plant or the part derived therefrom being characterized by having: a low climacteric peak on days 10-21 after harvest, development to ripening period at least 10% longer than a control, a shelf life increased by at least 7 days compared to a control, a reduced decay % compared to a control, or any combination thereof.

In some embodiments, the plant is a transgenic plant.

In some embodiments, the transgenic plant comprises at least one mutation in a cytosolic ICDH encoding gene.

In some embodiments, the control is a genetic reference plant comprising the same genotype of the transgenic plant and is lacking the at least one mutation selected agent reduces or prevents a climacteric peak on days 7-12 after harvest.

In some embodiments, the method further comprises a step of determining that the agent reduces or prevents a climacteric peak on days 7-12 after harvest.

### DESCRIPTION OF THE FIGURES

FIGS. 1A-1B are a graph and a micrograph showing the climacteric ripening of a tomato fruit that is characterized by a climax of respiration along with an increase in ethylene production (1A). Representative fruit from each phenotypic stage are presented (1B). IMG—Immature green; Mature green—MG; Breaker—B; Orange red—OR; and R—Red.

FIG. 2 is a vertical bar graph showing ICDHI mRNA levels in 1-MCP-treated and untreated Micro-Tom tomatoes. ICDH expression is reduced significantly in all ripening stages. Mature green—MG; Breaker—BRK; and Early orange—EO.

FIGS. 3A-3B are a 2-sequence alignment (3A) and an illustration of a partially sequence of a mutated ICDH1 protein (ICDH1_M; 3B). The generation of a CRISPR-based ICDH1 M mutant is presented. The addition of one nucleotide in ICDH1_M (Thymidine highlighted in underline and bold; 3A) creates a frame shift that introduces a stop codon 4 nucleotides downstream of the insertion site. The putative protein is less than half the size of the WT protein.

FIG. 4 is a vertical bar graph showing the total NADP-dependent ICDH activity of ICDH1_M compered to WT. ICDH activity was determined in fruits at different ripening stages. A decrease in activity was observed in all ripening stages. * Significant difference at the same ripening stage (p<0.05, n=3).

FIG. 5 is a micrograph showing shelf life of Micro Tom ICDH1_M after 50 days at room temperature. Upper row WT, lower row the mutant ICDH1 (ICDH1_M).

FIGS. 6A-6B are graphs showing ripening of ICDH1_M fruits and non-mutated fruits. (6A) Ethylene production rate. (6B) Respiration (represented as CO₂ production). Full line—non mutated, dashed line—ICDH1_M mutant.

FIG. 7 is horizontal charts showing the duration of ripening phases in wild type WT) and mutant (ICHD1_M) fruits. Fruits were harvested at the mature green stage and left to ripen at 20° C.

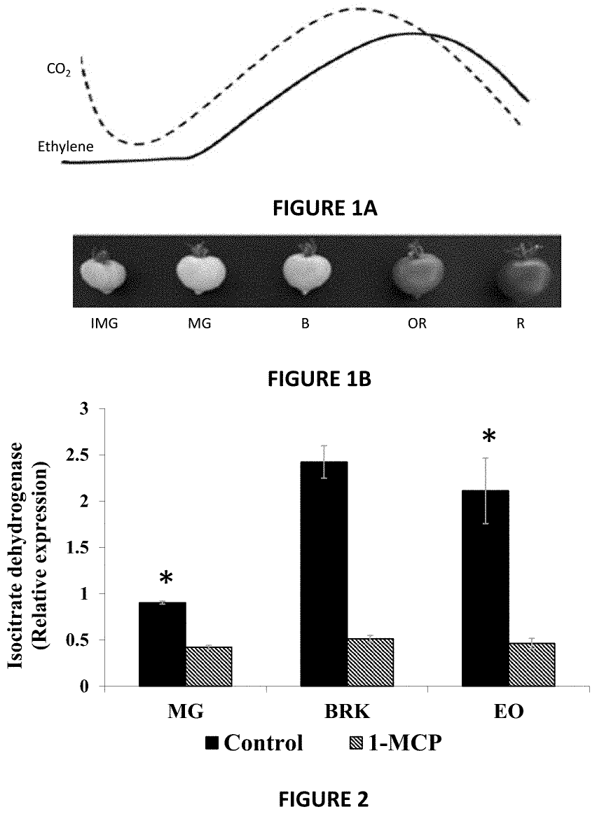

### DETAILED DESCRIPTION

The present invention, in some embodiments thereof, is based, in part, on the surprising findings that mutating a gene, in the respiratory pathway, e.g., ICDH1, affected not only respiration but also, ethylene production. Unlike other mutation (such as Rin and Nor), which are known to be active at very early stages of ripening, ICDH1 was found to be activated later and in a different biochemical pathway.

Further, the present invention, in some embodiments thereof, is based, in part, on the fact that the inactivated ICDH1, e.g., a mutated isoform thereof, prolonged ripening and did not holt it and did not have severe phenotypes, in sharp contrast to other mutations, as mentioned above, which are active at very early stages of ripening and may have negative effects, such as reducing quality taste, aroma and texture.

### Transgenic Plants and Parts Thereof

According to some embodiments, there is provided a transgenic plant comprising at least one mutation in an Isocitrate dehydrogenase (ICDH) encoding gene.

In some embodiments, a transgenic plant comprises at least one transgenic cell.

In some embodiments, the transgenic plant comprises at least one exogenous polynucleotide. In some embodiments, the exogenous polynucleotide comprises at least one mutation in an ICDH encoding gene.

In some embodiments, a plant part derived from the transgenic plant is provided.

In some embodiments, the ICDH is a cytosolic ICDH. As used herein, cytosolic ICDH encompasses any ICDH protein which is present and/or active in the cell cytoplasm. In some embodiments, cytosolic ICDH comprises any ICDH protein other than a mitochondrial ICDH.

Methods for determining the location of a protein, e.g., ICDH1, in a cell or a cell part or an organelle, or a tissue, or any combination thereof, are common and would be apparent to one of ordinary skill in the art. Non-limiting examples for methods of determining the location of a protein include, but are not limited to, immunoassays, such as immunohistochemistry, and immunocytochemistry using specific antibodies targeting, for example, ICDH, such as ICDH1.

In some embodiments, the transgenic plant has a reduced ethylene production rate. In some embodiments, the transgenic plant has a reduced respiration rate.

In some embodiments, reduced is by at least 5%, 10%, 20%, 30%, 40%, 50%, 60%, 70%, 80%, 90%, 95%, or 99%, or any value and range therebetween. Each possibility represents a separate embodiment of the invention. In some embodiments, reduced is by 1 to 70%, 5 to 90%, 10 to 100%, 15 to 85%, 20 to 65%, 5 to 45%, or 30 to 90%. Each possibility represents a separate embodiment of the invention.

In some embodiments, “reduced” and/or “increased” as used herein, are compared to a control.

In some embodiments, the term “control” as used herein, refers to any plant or a part derived therefrom comprising at least one cell devoid of the herein disclosed exogenous polynucleotide encoding the at least one mutated form of a cytosolic isocitrate dehydrogenase (ICDH) encoding gene.

In some embodiments, the control is a genetic reference plant. In some embodiments, the genetic reference plant comprises the same genotype of the transgenic plant and/but is lacking/lacks the at least one mutation. In some embodiments, the transgenic plant of the invention and the genetic reference plant comprise the same genome content, e.g., the same number of chromosomes harbouring the same nucleic acid sequences except the at least one mutation in a cytosolic ICDH encoding gene. In some embodiments, the genetic reference plant is devoid of the at least one mutation. In some embodiments, the genetic reference plant is devoid of the herein disclosed exogenous polynucleotide. In some embodiments, the genetic reference plant is devoid of an exogenous polynucleotide.

In some embodiments, a control is a non-transgenic plant, or a part derived therefrom. In some embodiments, a control is a wild type plant, or a part derived therefrom. In some embodiments, a control is a transgenic plant, or a part derived therefrom comprising an intact or a non-mutated cytosolic ICDH gene and/or polypeptide. In some embodiments, a control is a transgenic plant, or a part derived therefrom comprising a mutated cytosolic ICDH gene encoding an ICDH polypeptide having NADP-dependent ICDH activity equivalent or superior to the NADP-dependent ICDH activity of an intact or a wild type ICDH polypeptide. In some embodiments, a control is a transgenic plant, or a part derived therefrom comprising an intact or a non-mutated ICDH1 gene and/or protein. In some embodiments, a control is a transgenic plant, or a part derived therefrom comprising an intact or a non-mutated ICDH2cyt gene and/or protein. In some embodiments, a control is a transgenic plant, or a part derived therefrom comprising an intact or a non-mutated ICDH11 gene and/or protein.

As used herein, “ICDH activity” refers to the catalysation of the oxidative decarboxylation of isocitrate by the Isocitrate dehydrogenase enzyme, a process which yields alpha-ketoglutarate and CO₂. As used herein, ICDH activity refers to the reaction occurring outside the context of the citric acid cycle. In some embodiments, ICDH activity refers to the reaction that utilizes NADP⁺ as a cofactor. In some embodiments, ICDH activity refers to the reaction that does not utilize NAD⁺ as a cofactor. In some embodiments, ICDH activity refers to the reaction that is localized to the cytosol. In some embodiments, ICDH activity refers to the reaction that is localized to the peroxisome. In some embodiments, ICDH activity refers to the reaction that is not localized to the mitochondrion.

Methods for determining ethylene production rate, and respiration rate, are common and would be apparent to one of ordinary skill in the art. Such methods include, but are not limited to chromatography, such as gas chromatography (GC), as exemplified herein below.

In some embodiments, a mutated form of a cytosolic ICDH has a reduced NADP-dependent ICDH activity compared to a control ICDH. In some embodiments, a mutated form of a cytosolic ICDH is inactive (e.g., NADP-dependent activity).

Methods for determining NADP-dependent ICDH activity are exemplified herein below.

In some embodiments, the at least one cytosolic ICDH encoding gene is selected from: ICDH1, ICDH2cyt, ICDH11, or/and any combination thereof.

In some embodiments, the at least one cytosolic ICDH encoding gene is ICDH1.

In some embodiments, the exogenous polynucleotide encodes a mutated or a mutant plant cytoplasmic ICDH polypeptide, wherein the polynucleotide sequence is at least 50%, at least 65%, at least 70%, at least 75%, at least 80%, at least 85%, at least 90%, at least 95%, at least 96%, at least 97%, at least 98%, or at least 99%, homologous to a polynucleotide encoding a plant cytoplasmic ICDH, as long as the mutated or a mutant plant cytoplasmic ICDH polypeptide has reduced NADP-dependent ICDH activity compared to control (e.g., wild type ICDH), or is inactive. In some embodiments, the exogenous polynucleotide comprises an inserted nucleotide compared to the wild type ICDH encoding gene. In some embodiments, the exogenous polynucleotide misses or is devoid of one or two nucleotides compared to the wild type ICDH encoding gene. In some embodiments, wherein the exogenous polynucleotide misses or is devoid of two nucleotides compared to the wild type ICDH encoding gene, the two nucleotides can be missing continuously to one another, or in two distinct locations or positions along the exogenous polynucleotide compared to the wild type ICDH encoding gene. In some embodiments, the exogenous polynucleotide comprises a frameshift compared to the wild type ICDH encoding gene. In some embodiments, the exogenous polynucleotide encodes a shorter ICDH polypeptide compared to the wild type ICDH. In some embodiments, the exogenous polypeptide encodes an ICDH polypeptide harboring or comprising a premature stop codon, compared to the wild type ICDH polypeptide.

In some embodiments, the plant is Solanum lycopersicum (i.e., tomato). In some embodiments, the plant is Malus domestica (i.e. apple). In some embodiments, the plant is Solanum annum (i.e., pepper). In some embodiments, the plant is Solanum melongena (i.e., eggplant). In some embodiments, the plant is Solanum tuberosum (i.e., potato). In some embodiments, the plant is Musa acuminate (i.e., banana). In some embodiments, the plant is Fragaria vesca (i.e., strawberry). In some embodiments, the plant is Nicotiana tabacum (i.e., tobacco). In some embodiments, the plant is Arabidopsis thaliana.

In some embodiments, a transgenic Arabidopsis thaliana comprises a mutated form of the cytoplasmic ICDH polypeptide (AT1G65930) comprising the amino acid sequence: MAFEKIKVANPIVEMDGDEMTRVIWKSIKDKLITPFVELDIKYFDLGLPHRDATDDKVTI ESAEATKKYNVAIKCATITPDEGRVTEFGLKQMWRSPNGTIRNILNGTVFREPIICKNVP KLVPGWTKPICIGRHAFGDQYRATDAVIKGPGKLTMTFEGKDGKTETEVFTFTGEGGVA MAMYNTDESIRAFADASMNTAYEKKWPLYLSTKNTILKKYDGRFKDIFQEVYEASWKS KYDAAGIWYEHRLIDDMVAYALKSEGGYVWACKNYDGDVQSDFLAQGFGSLGLMTS VLVCPDGKTIEAEAAHGTVTRHFRVHQKGGETSTNSIASIFAWTRGLAHRAKLDDNAKL LDFTEKLEAACVGTVESGKMTKDLALIIHGSKLSRDTYLNTEEFIDAVAAELKERLN (SEQ ID NO: 1), wherein the mutated form comprises at least stop codon compared to an amino acid in that location in SEQ ID NO: 1, and wherein the mutated form has reduced NADP-dependent ICDH activity compared to SEQ ID NO: 1, or a control, or is inactive.

In some embodiments, a transgenic Fragaria vesca comprises a mutated form of the cytoplasmic ICDH polypeptide (XP_004309437) comprising the amino acid sequence: MAFEKIKVANPIVEMDGDEMTRIFWQSIKDKLILPFLDLDIKYFDLGLTHRDATDDKVTI ESAEATLKYNVAIKCATITPDEARMKEFNLKQMWRSPNGTIRNILNGTVFREPILCKNIP RLIPGWTKPICIGRHAFGDQYRATDTVIKGPGKLKLMFVPEGQDEKMEFEVFNFTGEGG VAIAMYNTDESIRAFAEASMNTAYEKKWPLYLSTKNTILKKYDGRFKDIFQEVYEAHW KSKYEAAGIWYEHRLIDDMVAYALKSEGGYVWACKNYDGDVQSDFLAQGFGSLGLM TSVLVCPDGKTIEAEAAHGTVTRHYRVHQRGGETSTNSIASIFAWTRGLAHRAKLDDNA KLLEFTQKLEEACIGTVESGKMTKDLALILHGPKLARNHYLNTEEFIDAVAAELKAKLC (SEQ ID NO: 2), wherein the mutated form comprises at least stop codon compared to an amino acid in that location in SEQ ID NO: 2, and wherein the mutated form has reduced NADP-dependent ICDH activity compared to SEQ ID NO: 2, or a control, or is inactive.

In some embodiments, a transgenic Malus domestica comprises a mutated form of the cytoplasmic ICDH polypeptide (XP_028952907) comprising the amino acid sequence: MAFQKIKVANPIVEMDGDEMTRVFWKSIKDKLILPFVELDIKYFDLGLPHRDATDDKVT VESAEATLKYNVAIKCATITPDEARMKEFSLKSMWRSPNGTIRNILNGTVFREPILCKNIP RLIPGWTKPICIGRHAFGDQYRATDAVIKGPGKLKLVFVPEGKDEKTELDVYDFTGEGG VALAMYNTDESIRAFAEASMTTAYEKKWPLYLSTKNTILKKYDGRFKDIFQEVYEANW KSKFDAAGIWYEHRLIDDMVAYALKSDGAYVWACKNYDGDVQSDMLAQGFGSLGLM TSVLVCPDGKTIEAEAAHGTVTRHYRVHQKGGETSTNSIASIFAWTRGLAHRAKLDDNA RLLEFTQKLEEACIGTVESGKMTKDLALILHGSKLARNHYLNTEEFIDAVAEELKAKLA C (SEQ ID NO: 3), wherein the mutated form comprises at least stop codon compared to an amino acid in that location in SEQ ID NO: 3, and wherein the mutated form has reduced NADP-dependent ICDH activity compared to SEQ ID NO: 3, or a control, or is inactive.

In some embodiments, a transgenic Nicotiana tabacum comprises a mutated form of the cytoplasmic ICDH polypeptide (XP_016443611) comprising the amino acid sequence: MAFDKIKVENPIVEMDGDEMTRVIWKSIKDKLICPFLELDIKYFDLGLPHRDATDDKVT VESAEATQKYNVAIKCATITPDEARVKEFNLKSMWRSPNGTIRNILNGTVFREPIMCKNI PRLVPGWTKPICIGRHAFGDQYRATDTVIQGAGKLKLVFVPEGTDEKTEFEVYNFTGAG GVALSMYNTDESIRSDFAEASMNMAYQKKWPLYLSTKNTILKKYDGRFKDIFQEVYEAN WKSKYEEAGIWYEHRLIDDMVAYALKSEGGYVWACKNYDGDVQSDFLAQGFGSLGL MTSVLVCPDGKTIEAEAAHGTVTRHYRVHQKGGETSTNSIASIFAWTRGLAHRATLDN NERLLDFTEKLEAACIGAVESGKMTKDLALIIHGSKLSRDHYLNTEEFIDAVADELKARL LKAKA (SEQ ID NO: 4), wherein the mutated form comprises at least stop codon compared to an amino acid in that location in SEQ ID NO: 4, and wherein the mutated form has reduced NADP-dependent ICDH activity compared to SEQ ID NO: 4, or a control, or is inactive.

In some embodiments, a transgenic Solanum annum comprises a mutated form of the cytoplasmic ICDH polypeptide (CA08g01590) comprising the amino acid sequence: MAFQKINVQNPIVEMDGDEMTRVIWKSIKDKLILPFLELDIKYFDLGLPHRDLTDDKVT VESAEATLKYNVAIKCATITPDEARVKEFKLKSMWRSPNGTIRNILNGTVFREPIMCKNI PRLVPGWTKPICIGRHAFGDQYRATDAVIQGAGKLKLVFVPEGSDEKTEYEVYNFTGAG GVALSMYNTDESIRAFADASMNMAYQKKWPLYLSTKNTILKKYDGRFKDIFQEVYEAS WKSKYEEAGIWYEHRLIDDMVAYALKSEGGYVWACKNYDGDVQSDFLAQGFGSLGL MTSVLVCPDGKTIEAEAAHGTVTRHYRVHQKGGETSTNSIASIFAWTRGLAHRATLDK NERLLDFTEKLEAACIGAVESGKMTKDLALIIHGSKLSREHYLNTEEFIDAVADELKAKL LKAKA (SEQ ID NO: 5), wherein the mutated form comprises at least stop codon compared to an amino acid in that location in SEQ ID NO: 5, and wherein the mutated form has reduced NADP-dependent ICDH activity compared to SEQ ID NO: 5, or a control, or is inactive.

In some embodiments, a transgenic Solanum melongena comprises a mutated form of the cytoplasmic ICDH polypeptide (SMEL_001g153250) comprising the amino acid sequence: MAFQKIVVQNPIVEMDGDEMTRVIWKSIKDKLILPFLELDIKYFDLGLPHRDATDDKVT VESAEATLKYNVAIKCATITPDEARVKEFNLKSMWRSPNGTIRNILNGTVFREPIMCKNI PRLVPGWTKPICIGRHAFGDQYRATDTVIKGAGKLKLVFVPEGSGEKSELEVYNFTGAG GVALSMYNTDESIRSFAEASMNMAFQKKWPLYLSTKNTILKKYDGRFKDIFQEVYEAN WKSKYEEAGIWYEHRLIDDMVAYALKSEGGYVWACKNYDGDVQSDFLAQGFGSLGL MTSVLVCPDGKTIEAEAAHGTVTRHYRVHQKGGETSTNSIASIFAWTRGLAHRATLDK NERLLDFTEKLEAACIGAVESGKMTKDLALIIHGSKLSREHYLNTEEFIDAVADELKAKL LKAKA (SEQ ID NO: 6), wherein the mutated form comprises at least stop codon compared to an amino acid in that location in SEQ ID NO: 6, and wherein the mutated form has reduced NADP-dependent ICDH activity compared to SEQ ID NO: 6, or a control, or is inactive.

In some embodiments, a transgenic Solanum tuberosum comprises a mutated form of the cytoplasmic ICDH polypeptide (XP_006354121) comprising the amino acid sequence: MAFQKITVQNPIVEMDGDEMTRVIWKSIKDKLILPFLELDIKYFDLGLPHRDATDDKVT VESAEATQKYNVAIKCATITPDEARVTEFNLKSMWRSPNGTIRNILNGTVFREPIMCKNI PRLVPGWTKPICIGRHAFGDQYRATDTVIKGAGKLKLVFVPEGSDEKTEFEVYNFTGAG GVALSMYNTDESVRSFAEASMNMAFQKKWPLYLSTKNTILKKYDGRFKDIFQEVYEAN WKSKYEEAGIWYEHRLIDDMVAYALKSEGGYVWACKNYDGDVQSDFLAQGFGSLGL MTSVLVCPDGKTIEAEAAHGTVTRHYRVHQKGGETSTNSIASIFAWTRGLAHRATLDN NERLLDFTEKLEAACIGAVESGKMTKDLALIIHGSKLSREHYLNTEEFIDAVADELKARL LKAKA (SEQ ID NO: 7), wherein the mutated form comprises at least stop codon compared to an amino acid in that location in SEQ ID NO: 7, and wherein the mutated form has reduced NADP-dependent ICDH activity compared to SEQ ID NO: 7, or a control, or is inactive.

In some embodiments, a transgenic Solanum lycopersicum comprises a mutated form of the cytoplasmic ICDH polypeptide (Solyc01g005560) comprising the amino acid sequence: MAFQKIIVQNPIVEMDGDEMTRVIWKSIKDKLILPFLELDIKYFDLGLPHRDATDDKVTIE SAEATQKYNVAIKCATITPDEARVKEFNLKSMWRSPNGTIRNILNGTVFREPIMCKNIPR LVPGWTKPICIGRHAFGDQYRATDTVIKGAGKLKLVFVPEGSDEKTEFEVYNFTGAGGV ALSMYNTDESVRAFAEASMNMAFQKKWPLYLSTKNTILKKYDGRFKDIFQEVYEANW KSKYEEAGIWYEHRLIDDMVAYALKSEGGYVWACKNYDGDVQSDFLAQGFGSLGLMT SVLVCPDGKTIEAEAAHGTVTRHYRVHQKGGETSTNSIASIFAWTRGLAHRATLDNNER LLDFTEKLEAACIGAVESGKMTKDLALIIHGSKLSREHYLNTEEFIDAVADELKAKLLKA KA (SEQ ID NO: 8), wherein the mutated form comprises at least stop codon compared to an amino acid in that location in SEQ ID NO: 8, and wherein the mutated form has reduced NADP-dependent ICDH activity compared to SEQ ID NO: 8, or a control, or is inactive.

In some embodiments, a transgenic Musa acuminata comprises a mutated form of the cytoplasmic ICDH polypeptide (Ma04_p00870.1) comprising the amino acid sequence: MAFEKIKVFNPIVEMDGDEMTRVFWKSIKEKLIFPFLDLDIKYFDLGLPNRDATDDKVTI ESAEATLNYNVAIKCATITPDEARVKEFNLKSMWKSPNGTIRNILNGTVFREPIICKNIPR LVPGWTKPICIGRHAFGDQYRATDTVIKGPGKLKLVFEGKDEEVELEVFNFTGAGGVAL SMYNTDESIRAFADASMATAYQKKWPLYLSTKNTILKKYDGRFKDIFQEVYETEWKSK FEAAGIWYEHRLIDDMVAYALKSEGGYVWACKNYDGDVQSDFLAQGFGSLGLMTSVL VCPDGKTIEAEAAHGTVTRHYRVHQKGGETSTNSIASIFAWSRGLAHRAKLDDNARLL DFTEKLEAACVGTVESGKMTKDLALLIHGSSVTRAQYLNTEEFIDAVASELRARLSA (SEQ ID NO: 9), wherein the mutated form comprises at least stop codon compared to an amino acid in that location in SEQ ID NO: 9, and wherein the mutated form has reduced NADP-dependent ICDH activity compared to SEQ ID NO: 9, or a control, or is inactive.

In some embodiments, the herein disclosed method teaches the use of guide RNA specifically targeting an endogenous cytoplasmic ICDH encoding gene so as to introduce a premature stop codon thereto, e.g., any one of SEQ ID Nos.: 1-9, by contacting the plant or a part derived therefrom with the aforementioned gRNA, thereby obtaining a transgenic plant comprising an exogenous polynucleotide encoding a mutated cytoplasmic ICDH polypeptide.

Homology (e.g., percent homology) can be determined using any homology comparison software, including for example, the BlastP software of the National Center of Biotechnology Information (NCBI) such as by using default parameters.

Identity (e.g., percent homology) can be determined using any homology comparison software, including for example, the BlastN software of the National Center of Biotechnology Information (NCBI) such as by using default parameters.

In some embodiments, the exogenous polynucleotide encoding a mutated polypeptide as disclosed herein, is optimized for plant expression. The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681).

In some embodiments, a fruit derived is from the transgenic plant of the invention.

In some embodiments, the fruit is characterized by having a low climacteric peak. In some embodiments, a low climacteric peak is on days 15-21 after harvest, days 14-30 after harvest, days 16-28 after harvest, days 19-26 after harvest, days 15-25 after harvest, days 17-24 after harvest, or days 20-45 after harvest. Each possibility represents a separate embodiment of the invention.

In some embodiments, the fruit has a development to ripening period at least 5%, at least 10%, at least 15%, at least 30%, at least 50%, at least 100%, at least 250%, at least 500%, at least 750%, or at least 1,000% longer than a control, or any value and range therebetween. Each possibility represents a separate embodiment of the invention. In some embodiments, the fruit has a development to ripening period 5 to 250%, 25 to 350%, 50 to 650%, 100 to 250%, 250 to 850%, 450 to 925%, 325 to 1,000%, or 100 to 1,250% longer than a control. Each possibility represents a separate embodiment of the invention.

In some embodiments, the fruit has a shelf life increased by: at least 3 days, at least 4 days, at least 5 days, at least 6 days, at least 7 days, at least 9 days, at least 12 days, at least 15 days, at least 18 days, at least 21 days, at least 25 days, at least 28 days, at least 1 month, at least 2 months, or at least 3 months compared to control, or any value and range therebetween. Each possibility represents a separate embodiment of the invention. In some embodiments, the fruit has a shelf life increased by 3 to 21 days, 4 to 28 days, 10 to 26 days, 8 to 20 days, 15 to 35 days, 18 to 45 days, 2 to 4 weeks, 1 to 5 weeks, 3 to 8 weeks, 1 to 2 months, 1 to 3 months, 1 to 4 months, or 2 to 5 months, compared to control. Each possibility represents a separate embodiment of the invention.

The term “plant” as used herein encompasses whole plants, ancestors and progeny of the plants and plant parts, including fruits, seeds, leaves, shoots, stems, roots (including tubers), and plant cells, tissues and organs, of any species of woody, herbaceous, perennial or annual plant. The term “plant” also therefore encompasses suspension cultures, embryos, meristematic regions, callus tissue, leaves, gametophytes, sporophytes, pollen, and microspores. The term “plant” also refers to a plurality of plant cells that are largely differentiated into a structure that is present at any stage of a plant's development. Such structures include, but are not limited to, fruit, root, stem, shoot, leaf, flower, petal, etc.

In some embodiments, the plant part is a fruit.

As used herein, the term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 Kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The terms “polynucleotide”, “polynucleotide sequence”, and “nucleic acid sequence” are used interchangeably herein. These terms encompass nucleotide sequences and the like. A polynucleotide may be a polymer of RNA, DNA or hybrid thereof, that is single- or double-stranded, linear or branched, and that optionally contains synthetic, non-natural or altered nucleotide bases.

The term “recombinant” refers to an artificial combination of at least two otherwise separated segments of sequence, e.g., by chemical synthesis or by the manipulation of isolated segments of nucleic acids by genetic engineering techniques.

The term “construct” as used herein refers to an artificially assembled or isolated nucleic acid molecule which includes a gene of interest. In general, a construct may include a gene or genes of interest, a marker gene which in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. Transient transformation may be detected by, for example, enzyme-linked immunosorbent assay (ELISA), which detects the presence of a polypeptide encoded by one or more of the exogenous polynucleotides. Alternatively, transient transformation may be detected by detecting the activity of the protein (e.g. β-glucuronidase) encoded by the exogenous polynucleotide.

The term “transient transformant” refers to a cell which has transiently incorporated one or more exogenous polynucleotides. In contrast, the term “stable transformation” or “stably transformed” refers to the introduction and integration of one or more exogenous polynucleotides into the genome of a cell. Stable transformation of a cell may be detected by Southern blot hybridization of genomic DNA of the cell with nucleic acid sequences which are capable of binding to one or more of the exogenous polynucleotides. Alternatively, stable transformation of a cell may also be detected by enzyme activity of an integrated gene in growing tissue or by the polymerase chain reaction of genomic DNA of the cell to amplify exogenous polynucleotide sequences. The term “stable transformant” refers to a cell which has stably integrated one or more exogenous polynucleotides into the genomic or organellar DNA. It is to be understood that a plant or a plant cell transformed with the nucleic acids, constructs and/or vectors of the present invention can be transiently as well as stably transformed. The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

Plants that are useful in the methods of the invention include all plants which belong to the superfamily Viridiplantae, such as monocotyledonous and dicotyledonous plants which are of commercial value, including a fodder or forage legume, ornamental plant, food crop, tree, or shrub selected from the following non-limiting list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus deer, Baikiaea plurijuga, Betula spp., Brassica spp., Bruguiera gymnorrhiza, Burkea africana, Butea frondosa, Cadaba farinosa, Calliandra spp, Camella sinensis, Canna indica, Capsicum spp., Cassia spp., Cent roema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Ciyptomeria laponica, Cymbopogon spp., Cynthea dealbata, Cydonia oblonga, Dalbergia monetaria, Davalila divaricata, Desmodium spp., Dicksonia squarosa, Diheteropogon amplectens, Dioclea spp, Dolichos spp., Doiycnium rectum, Echinochloa pyramidalis, Ehrartia spp., Eleusine coracana, Era grestis spp., Erythrina spp., Eucalyptus spp, Euclea schimpen Eulalia villosa, Fagopyrum spp., Felloa sellowiana, Fr agar is spp., Flemingia spp, Freycinetia banksii, Geranium thunbergi, Ginkgo biloba, Glycine javanica, Gliricidia spp, Gossypium hirsutum, Gr e villea spp., Guibourtia coleosperma, Hedysarum spp., Hemarthia altissima, Heteropogon con tortus, Hordeum vulgare, Hyparrhenia rufa, Hypericum erectum, Hyper thelia dissoluta, Indigo incarnata, Iris spp., Jatropha curcas, Leptarrhena pyrolifolia, Lespediza spp., Lettuca spp., Leucaena leucocephala, Loudetia simplex, Lotonus bainesi, Lotus spp, Macrotyloma axifiare, Malus spp., Manihot esculenta, Medicago sativa, Metasequoia glyptostroboides, Musa sapientum, Nicotianum spp., Onobtychis spp., Ornithopus spp., Oryza spp., Peltophorum african urn, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria flecki, Pogonarthria squarrosa, Populus spp., Prosopis cineraria, Pseudotsuga menziesi, Pterolobium stellatum, Pyrus communis, Quercus spp., Rhaphiolepsis umbellata, Rhopalostylis sapida, Rhu.s natalensis, Ribes grossularia, Ribes spp., Robinia pseudoacacia, Rosa spp., Rub us spp., Salix spp., Schyzachyrium sanguineurn, Sciadopitys verticillata, Sequoia sempen'irens, Sequoiadendron giganteum, Sorghum bicolor, Spinacia spp., Sporobolus flmbriatus, Stiburus alopecuroides, Stylosanthos humilis, Tadehagi spp, Taxodium distichum, Themeda triandra, Trifollum spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively, algae and other non-Viridiplantae can be used.

Expressing the exogenous polynucleotide as disclosed herein within the plant can be performed by transforming one or more cells of the plant with the exogenous polynucleotide, followed by generating a mature plant from the transformed cells and cultivating the mature plant under conditions suitable for expressing the exogenous polynucleotide within the mature plant.

In some embodiments, transformation is affected by introducing to a plant cell a nucleic acid construct which includes the exogenous polynucleotide and at least one promoter capable of directing transcription of the exogenous polynucleotide in the plant cell. Further details of suitable transformation approaches are provided hereinbelow.

The terms “promoter”, “promoter element” or “promoter sequence” as used herein, refer to a DNA sequence that is located at the 5′ end (“precedes”) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. New promoters of various types useful in plant cells are constantly being discovered; numerous examples may be found in Okamuro J K and Goldberg R B (1989) Biochemistry of Plants 15:1-82.

Any suitable promoter sequence can be used, e.g., fruit specific promoter, flower specific promoter, and others. The following types of promoters are non-limiting examples of promoters used to over-express a selected gene: general promoter, root specific promoter, root-tips specific promoter, drought-induced root promoter, biotic stress-induced promoter, abiotic stress-induced promoter, nitrogen induced promoter, ammonium or nitrate induced promoter, phosphate fertilizer-induced promoter, leaf specific promoter, inducible promoter, constitutive promoter, promoter with two or more of the characteristics described above, or other novel promoters.

Further, fruit specific promoters are also applicable according to the present invention. Non-limiting examples for a fruit specific promoter include, but are not limited to E8, E4, TFM7, TFM9, 2A11, or others.

Further, flower specific promoters are also applicable according to the present invention. Non-limiting examples for a flower specific promoter include, but are not limited to EPSPS promoter, B gene promoter, PDS promoter, CHRC promoter, or others.

Choice of the promoter is based largely on the phenotype of interest and is determined by such factors as tissue (e.g., fruit, seed, root, pollen, vascular tissue, flower, carpel, etc.), inducibility (e.g., in response to wounding, heat, cold, drought, light, pathogens, etc.), timing, developmental stage, and the like. Numerous known promoters have been characterized and can favorably be employed to promote expression of an exogenous polynucleotide as disclosed herein in a transgenic plant or cell of interest.

Measures should be taken, however, to select a promoter which will mediate desirable expression levels of the transgene so as to avoid reallocating excessive energetic resources which may affect final yield, strength, mass and lodging and incidence of foliar pathogens. This should also be viewed from an economic perspective.

Suitable constitutive promoters include, for example, CaMV 35S promoter (Odell et al, Nature 313:810-812, 1985); Arabidopsis At6669 promoter (PCT No WO2004/104162); TT105 (PCT NO.: WO2004/081173) maize Ubi 1 (Christensen et al, Plant Sol. Biol. 18:675-689, 1992); rice actin (McElroy et al, Plant Cell 2:163-171, 1990); pEMU (Last et al., Theor. Appl. Genet. 81:581-588, 1991); and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142. Suitable tissue-specific promoters include, but not limited to, leaf-specific promoters such as described, for example, by Yamamoto et ah, Plant J. 12:255-265, 1997; Kwon et al, Plant Physiol. 105:357-67, 1994; Yamamoto et al, Plant Cell Physiol. 35:773-778, 1994; Gotor et al, Plant J. 3:509-18, 1993; Orozco et al, Plant MoL Biol. 23:1129-1138, 1993; and Matsuoka et al, Proc. Natl. Acad. Sci. USA 90:9586-9590, 1993. Root promoters include, but are not limited to, the ROOTP promoter (Upstream region of the gene ATXTH19 (AT4G30290, Xyloglucan endotransglucosylase/hydrolase 19, described in Vissenberg K, et al. Plant Cell Physiol. 2005 January; 46(1): 192-200).

A variety of plant gene promoters are known to regulate gene expression in response to environmental, hormonal, chemical, developmental signals, and in a tissue-active manner. Examples of seed-specific promoters (such as the napin, phaseolin or DC3 promoter described in U.S. Pat. No. 5,773,697), fruit-specific promoters that are active during fruit ripening, such as the dru 1 promoter (U.S. Pat. No. 5,783,393), or the 2Al 1 promoter (U.S. Pat. No. 4,943,674) and the tomato polygalacturonase promoter (Bird et al. (1988) Plant Mol. Biol. 11: 651-662), root-specific promoters, such as ARSK1, and those disclosed in U.S. Pat. Nos. 5,618,988, 5,837,848 and 5,905,186, epidermis-specific promoters, including CUT1 (Kunst et al. (1999) Biochem. Soc. Tians. 28: 651-654), pollen-active promoters such as PTA29, PTA26 and PTA 13 (U.S. Pat. No. 5,792,929), promoters active in vascular tissue (Ringli and Keller (1998) Plant Mol. Biol. 37: 977-988), flower-specific (Kaiser et al. (1995) Plant Mol. Biol. 28: 231-243), pollen (Baerson et al. (1994) Plant MoL Biol. 26: 1947-1959), carpels (Ohl et al. (1990) Plant Cell 2: 837-848), pollen and ovules (Baerson et al. (1993) Plant Mol. Biol. 22: 255-267), auxin-inducible promoters (such as that described in van der Kop et al. (1999) Plant Mol. Biol. 39: 979-990 or Baumann et al. (1999) Plant Cell 11: 323-334), cytokinin-inducible promoter (Guevara-Garcia (1998) Plant Mol. Biol. 38: 743-753), promoters responsive to gibberellin (Shi et al. (1998) Plant Mol. Biol. 38: 1053-1060, Willmott et al. (1998) Plant Mol. Biol. 38: 817-825) and the like. Additional promoters are those that elicit expression in response to heat (Ainley et al. (1993) Plant Mol. Biol. 22: 13-23), light (e.g., the pea rbcS-3A promoter, described in Kuhlemeier et al. (1989) Plant Cell 1: 471-478, and the maize rbcS promoter, described in Schaffier and Sheen (1991) Plant Cell 3: 997-1012); wounding (e.g., wunl, described in Siebertz et al. (1989) Plant Cell 1: 961-968), pathogens (such as the PR-I promoter described in Buchel et al. (1999) Plant Mol. Biol. 40: 387-396, and the PDF1.2 promoter described in Manners et al. (1998) Plant MoI. Biol. 38: 1071-1080), and chemicals such as methyl jasmonate or salicylic acid (Gatz (1997) Annu. Rev. Plant Physiol. Plant MoI. Biol. 48: 89-108). In addition, the timing of the expression can be controlled by using promoters such as those acting at senescence (Gan and Amasino (1995) Science 270: 1986-1988); or late seed development (Odell et al. (1994) Plant Physiol. 106: 447-458).

Other examples of promoters are a SUC2 promoter (Truernit and Sauer, Planta. (1995) 196:564-70), and a stress-inducible promoter such as RD29A (Yamaguchi-Shinozaki and Shinozaki K. Plant Cell (1994) 6:251-264), promoters from the PlantProm database (Shahmuradov et al. Nucleic Acids Res. (2003) 31: 114-7), the rice CatB promoter (Iwamoto et al. Plant Physiol Biochem. (2004) 42:241-9), the root specific and phosphate-deficiency inducible barley promoters of the phosphate transporter gene family (HvPhtl;1 and HvPhtl;2) (Schunmann et al. (2004); 55:855-65), tissue specific and constitutive promoters illustrated in patent No: WO2004/081173, in patent No: U.S. Pat. No. 5,633,363, in patent No: WO 2000/15662, in patent No: WO 2004/013169, in patent No: US 2005/010974, in patent No: WO 2005/035770, in patent No: US 2001/0047525, in patent No: U.S. Pat. No. 5,837,848, in patent No: U.S. Pat. No. 6,018,099, etc.

In some embodiments, a nucleic acid construct comprising the exogenous polynucleotide further comprises an appropriate selectable marker and/or an origin of replication. In some embodiments, the nucleic acid construct is a shuttle and/or an expression vector. In some embodiments, the vector comprises a prokaryote origin of replication, an eukaryote origin of replication, or both. In some embodiments, the vector can propagate in E. coli (wherein the construct comprises an appropriate selectable marker and origin of replication) and be compatible with propagation in cells. The construct according to the present invention can be, for example, a plasmid, a bacmid, a phagemid, a cosmid, a phage, a virus or an artificial chromosome.

In some embodiments, the nucleic acid construct can be utilized to stably or transiently transform plant cells. In some embodiments, in stable transformation, the exogenous polynucleotide is integrated into the plant genome and as such it represents a stable and inherited trait. In some embodiments, in transient transformation, the exogenous polynucleotide is expressed by the cell transformed but it is not integrated into the genome and as such it represents a transient trait.

In some embodiments, a stable transformation may include the integration of an RNA interfering encoding polynucleotide (RNAi). In some embodiments, the RNAi encoding polynucleotide is permanently integrated into the genome of the plant. In some embodiments, the RNAi encoding polynucleotide is expressed in an inducible manner. In some embodiments, the RNAi encoding polynucleotide is expressed in a constitutive manner. Both inducible and constitutive manners of expression are under the regulation of a compatible promoters as disclosed herein above.

There are various methods of introducing foreign genes into both monocotyledonous and dicotyledonous plants (Potrykus, L, Annu. Rev. Plant. Physiol., Plant. MoI. Biol. (1991) 42:205-225; Shimamoto et al, Nature (1989) 338:274-276). The principle methods of causing stable integration of exogenous DNA into plant genomic DNA include two main approaches: (i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112. (ii) Direct DNA uptake: Paszkowski et al, in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6: 1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al Bio/Technology (1988) 6:559-563; McCabe et al Bio/Technology (1988) 6:923-926; Sanford, Physiol. Plant. (1990) 79:206-209; by the use of micropipette systems: Neuhaus et al, Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. ScL USA (1986) 83:715-719. The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues. Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different, and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment. Preferably, mature transformed plants generated as described above are further selected for the trait of interest (e.g., prolonged ripening, reduced pathogen susceptibility, etc.). Examples of such screening assays are provided hereinbelow. Thus, for example, transgenic plants may be screened for an improved trait, e.g., prolonged ripening under normal or stress conditions as will be further described hereinbelow. Alternatively, or additionally, transformed and non-transformed (wild type) plants are exposed to a biotic stress condition, such as a pathogen inoculation, e.g., fungal pathogen (such as Botrytis). Following exposure to the stress condition the plants are frequently monitored until substantial physiological and/or morphological effects appear in wild type plants. Subsequently, transformed plants not exhibiting substantial physiological and/or morphological effects, or exhibiting higher biomass than wild-type plants, are identified as biotic stress, e.g., fungal pathogen, tolerant or resistant plants.

In some embodiments, transformation is a transient transformation of a leaf cell, a meristematic cell or of the whole plant. Transient transformation can be affected by any of the direct DNA transfer methods described herein above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261. Preferably, the virus of the present invention is avirulent and thus is incapable of causing severe symptoms such as reduced growth rate, mosaic, ring spots, leaf roll, yellowing, streaking, pox formation, tumor formation and pitting. A suitable avirulent virus may be a naturally occurring avirulent virus or an artificially attenuated virus. Virus attenuation may be effected by using methods well known in the art including, but not limited to, sub-lethal heating, chemical treatment or by directed mutagenesis techniques such as described, for example, by Kurihara and Watanabe (Molecular Plant Pathology 4:259-269, 2003), Gal-on et al. (1992), Atreya et al. (1992) and Huet et al. (1994).

In some embodiments, the at least one exogenous polynucleotide molecule encodes at least one component of the CRISPR-Cas system. In some embodiments, the at least one component of the CRISPR-Cas system is a guide RNA or a Cas protein. In some embodiments, the Cas is Cas9.

As used herein, the term “gRNA” comprises any gRNA which specifically hybridizes with a cytoplasmic ICDH encoding gene and in turn induces the mutagenesis of the cytoplasmic ICDH encoding gene. In some embodiments, the resulting mutated cytoplasmic ICDH gene encodes a protein product having reduced activity or is inactivated, as disclosed herein. As used herein, the term “specifically” refers to that the gRNA does not target mitochondrial IDH genes, other genes, other sequences in the genome of the plant, or any combination thereof. As used herein, the term “specifically” refers to that the gRNA has no off-targeting effects. A person of ordinary skill in the art would appreciate that off-targeting effects include, but are not limited to, adverse effects, limited viability, inferior traits, or any combination thereof, and as such should be avoided. Methods for designing gRNA targeting specific sites which would induce high hybridization (and subsequent gene editing) while avoiding off-targeting effects (e.g., hybridization to other non-related regions in the genome), would be apparent to one of ordinary skill in the art. Non-limiting examples for such methods, include bioinformatics using designated algorithms, such as CRISPRdirect, as described by Naito et al., 2015 (Bioinformatics. 31(7): 1120-1123).

In some embodiments, the gRNA comprises the nucleic acid sequence 5′-CCATTCTTAAGAAATATGATGGGAGG-3′ (SEQ ID NO: 10). In some embodiments, the gRNA consists of the nucleic acid sequence 5′-CCATTCTTAAGAAATATGATGGGAGG-3′ (SEQ ID NO: 10).

In general, a CRISPR-Cas or CRISPR system as used in the foregoing documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA (crRNA) and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). When the CRISPR protein is a C2c2 protein, a tracrRNA is not required.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise DNA or RNA polynucleotides. The term “target DNA or RNA” refers to a DNA or RNA polynucleotide being or comprising the target sequence. In other words, the target DNA or RNA may be a DNA or RNA polynucleotide or a part of a DNA or RNA polynucleotide to which a part of the gRNA, i.e. the guide sequence, is designed to have complementarity and to which the effector function mediated by the complex comprising CRISPR effector protein, and a gRNA is to be directed. In some embodiments, a target sequence is located in the nucleus or cytoplasm of a cell. In some embodiments, the target is a plant gene.

As used herein, the term “crRNA” or “guide RNA” or “single guide RNA” or “sgRNA” or “one or more nucleic acid components” refers to a polynucleotide comprising any polynucleotide sequence having sufficient complementarity with a target nucleic acid sequence to hybridize with the target nucleic acid sequence and to direct sequence-specific binding of a RNA-targeting complex comprising the gRNA and a CRISPR effector protein, to the target nucleic acid sequence. In general, a gRNA may be any polynucleotide sequence (i) being able to form a complex with a CRISPR effector protein and (ii) comprising a sequence having sufficient complementarity with a target polynucleotide sequence to hybridize with the target sequence and direct sequence-specific binding of a CRISPR complex to the target sequence. As used herein, the term “capable of forming a complex with the CRISPR effector protein” refers to the gRNA having a structure that allows specific binding by the CRISPR effector protein to the gRNA such that a complex is formed that is capable of binding to a target DNA in a sequence specific manner and that can exert a function on said target DNA. Structural components of the gRNA may include direct repeats and a guide sequence (or spacer). The sequence specific binding to the target DNA is mediated by a part of the gRNA, the “guide sequence”, being complementary to the target DNA. In some embodiments, the term guide RNA, i.e. RNA capable of guiding Cas to a target locus, is used as in foregoing cited documents such as WO2014/093622 (PCT/US2013/074667). As used herein, the term “wherein the guide sequence is capable of hybridizing” refers to a subsection of the gRNA having sufficient complementarity to the target sequence to hybridize thereto and to mediate binding of a CRISPR complex to the target DNA. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, or 99%, or any value and range therebetween. Each possibility represents a separate embodiment of the invention. In some embodiments, the degree of complementarity between a guide sequence and its corresponding target sequence, when optimally aligned using a suitable alignment algorithm, is 100%. In some embodiments, the degree of complementarity between a guide sequence and its corresponding target sequence, when optimally aligned using a suitable alignment algorithm, is 50-95%, 60-100%, 65-95%, 70-80%, 55-85%, or 85-100%. Each possibility represents a separate embodiment of the invention. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is at least: 5, 10, 11, 12, 13, 14, 15, 16, 17, 18, 19, 20, 21, 22, 23, 24, 25, 26, 27, 28, 29, 30, 35, 40, 45, 50, or 75 nucleotides in length, or any value and range therebetween. Each possibility represents a separate embodiment of the invention. In some embodiments, a guide sequence is 5-75 nucleotides in length, 15-35 nucleotides in length, 25-55 nucleotides in length, 5-45 nucleotides in length, 10-35 nucleotides in length, or 10-45 nucleotides in length. Each possibility represents a separate embodiment of the invention. In some embodiments, the guide sequence is 10 to 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, mutating a target DNA may be evaluated by sequencing the targeted sequence, Other assays are possible, and will be apparent to those skilled in the art of molecular biology.

Other guidance for applying CRISPR methodology in plants and/or cells derived therefrom can be found in, for example, Belanto et al., (2017; WO2018107103A1).

In some embodiments, the at least one polynucleotide is an inhibitory polynucleotide molecule.

In some embodiments, the inhibitory polynucleotide molecule is an interfering RNA (RNAi).

In some embodiments, the inhibitory polynucleotide is selected from: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

As used herein, the term “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein, the term “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

As used herein, the terms “small interfering RNA” or “siRNA” refers to any small RNA molecule capable of inhibiting or down regulating gene expression by mediating RNA interference in a sequence specific manner. The small RNA can be, for example, from 18 to 21 nucleotides long.

In some embodiments, the RNAi molecule is selected from: small interfering RNA (siRNA), short hairpin RNA (shRNA), and double stranded RNA (dsRNA).

### Methods

According to some embodiments, the method of the invention is for reducing ethylene production rate, respiration rate, or both, in a plant or a part derived therefrom.

According to some embodiments, the method of the invention is for increasing the shelf life of a plant or a part derived therefrom.

In some embodiments, the method comprises a step of contacting the plant or a part derived therefrom with an effective amount of an agent, wherein the agent is capable of reducing cytosolic ICDH activity in the plant or a plant part derived therefrom.

In some embodiments, there is provided a composition comprising the agent and a compatible or acceptable excipient, diluent, or carrier. In some embodiments, the excipient, diluent, or a carrier is an agriculturally compatible or acceptable carrier.

In some embodiments, the contacting is pre-harvest contacting. In some embodiments, the contacting is post-harvest contacting. In some embodiments, the contacting is pre-harvest and post-harvest contacting.

According to the method of the invention, in some embodiments thereof, contacting results in a plant or a part derived therefrom being characterized by having: a low climacteric peak, development to ripening period at least 10% longer than a control, a shelf life increased by at least 7 days compared to control, a reduced decay % compared to control, or any combination thereof.

In some embodiments, the method comprises reducing cytosolic NADP-dependent ICDH activity in a transient manner (e.g., transiently reducing). In some embodiments, the method comprises reducing cytosolic NADP-dependent ICDH activity in constitutive manner (e.g., constitutively reducing). In some embodiment, transiently refers to an event that is confined in time, e.g., not permanently or constitutively. In some embodiments, transiently is by means of an induction. In some embodiments, transiently refers to a case wherein the effect is not inherited to a progeny or a subsequent or a derived organism.

As used herein, constitutively refers to ongoing, continuous. As used herein, constitutive or permanent refers to a case wherein the effect is inherited or transmitted to a progeny, germ cells, seeds, or a subsequent or a derived organism.

According to some embodiments, there is provided a method for producing an agent suitable for increasing shelf life of a plant or a part derived therefrom, comprising: obtaining an agent that reduces cytosolic NADP-dependent ICDH activity, determining the ethylene production rate, respiration rate, or both in the presence of the obtained agent, and selecting at least one agent the reduces ethylene production rate, respiration rate, or both, thereby producing an agent suitable for increasing the shelf life of a plant or a part derived therefrom.

In some embodiments, the selected agent reduces or prevents a climacteric peak on days 7-12 after harvest, 7-15 after harvest, 9-20 after harvest, 8-14 after harvest, 11-25 after harvest, 6-16 after harvest, or 5-14 after harvest. Each possibility represents a separate embodiment of the invention.

In some embodiments, the method further comprises a step of determining that the agent reduces or prevents a climacteric peak on days 7-12 after harvest, 7-15 after harvest, 9-20 after harvest, 8-14 after harvest, 11-25 after harvest, 6-16 after harvest, or 5-14 after harvest. Each possibility represents a separate embodiment of the invention.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Plant Material and 1-MCP Treatment

Tomato (Solanum lycopersicum; cv. Micro Tom) plants were grown under natural light in a climate-controlled greenhouse, at 24° C. during the day and 18° C. during the night. 1-MCP was applied using SmartFresh powder (Rohm and Haas, Philadelphia, Pa., USA) containing 0.14% 1-MCP as active ingredient, dissolved in warm water at a concentration of 2,000 nL·mL⁻¹ 1-MCP (stock solution). Fruits were enclosed in a 30 L airtight barrel and the stock solution was injected to a final concentration of nL·mL⁻¹ 1-MCP. Treatment duration was 24 hr at 20° C. For the control, fruits were treated identically excluding 1-MCP application (Gamrasni et al., 2017).

### RNA Extraction and Quantitative Real Time-PCR Assay

RNA was extracted using NucleoSpin® RNA Plant kit (Macherey-Nagel) according to the manufacturer's protocol. cDNA was prepared from 1 μg total RNA with Verso cDNA Synthesis Kit (ThermoFisher Scientific). The reaction was performed at 42° C. for 30 min. followed by inactivation at 95° C. for 2 min. Quantitative Real Time-PCR (qRT-PCR) was performed on a StepOnePlus™ system (Applied Biosystems™) using syber green mix (SYBR™ Select Master Mix (Applies Biosystems™) and ICDH gene specific oligonucleotides for amplification of 150-200 bp long fragments. The amplification cycle included an initial denaturation step of 20 s at 95° C., followed by 40 cycles of 3 s at 95° C., 30 s at the relevant annealing temperature and 30 s at 72° C. (ICDH-F2 ACAACGAACGGCTCTTGGAT (SEQ ID NO: 11) and ICDH-R2 TGAGCTCATCAGCTACAGCG (SEQ ID NO: 12) annealing temperature 60° C.; S1ASR-F2 CCTGTTCCACCACAAGGACAA (SEQ ID NO: 13) and S1ASR-R2 TGAGCTCATCAGCTACAGCG (SEQ ID NO: 14) annealing temperature 63° C. Relative expression ratio was normalized to the S1ASR gene (Solyc04g071610) used as an internal control (Relative RNA levels were determined by the delta-delta Ct method). The significance of results (n=3) was evaluated by independent t-test at *P<0.05 using the SPSS Statistics 20 software (IBM, USA).

### Gas Chromatography Analysis of Ethylene and CO₂ Production

Immediately after harvest, fruits were placed in a storage room at 20° C. with 70% relative humidity. Ethylene and CO₂ were measured daily from individual fruits enclosed in 30 mL tubes for 2 hr at 20° C. Two 2.5 mL samples were collected with a syringe from the headspace of each tube. For ethylene assay, samples were injected into a Varian 300 gas chromatograph (GC) equipped with Alumina column and Flame Ionization Detector (FID). Temperatures of the injector, column and detector were 85° C., 75° C., and 65° C. respectively. Ethylene production was expressed as pmole·kg⁻¹·s⁻¹.

Respiration—CO₂ was determined using a GOW-MAC 850 GC equipped with a Poropak column and a thermal conductivity detector (TCD). Column temperature was 54° C. and the injector and detector were held at room temperature. CO₂ production was expressed as mg·kg⁻¹·h⁻¹. Helium was the carrier gas for both chromatographs.

### Example 1

### CRISPR-Cas9-Mutated Solanum lycopersicum ICDH1 Prolongs Fruit Shelf-Life

Tomato fruits were exposed to the ethylene inhibitor 1-Methylcyclopropene (1-MCP) and the mRNA production of ICDH1 determined using Real time PCR. Although ICDH1 is part of the respiratory pathway its mRNA production was reduced significantly indicating that transcription factors induced by ethylene regulate ICDH1 expression (FIG. 2).

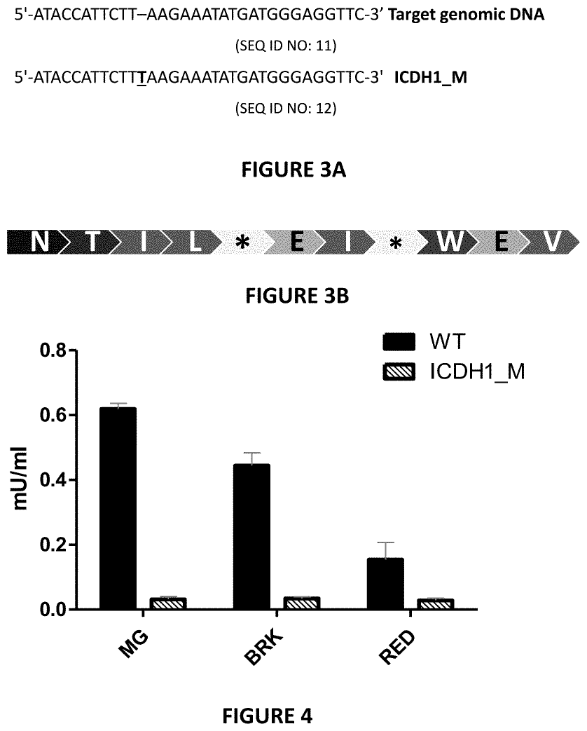

The inventors suggested that mutating specifically cytoplasmic ICDHs would be needed so as to block/inhibit ICDH1 and subsequently prolong ripening period. The inventors used the CRISPR-Cas9 system with a gRNA comprising the sequence: 5′-CCATTCTTAAGAAATATGATGGGAGG-3′ (SEQ ID NO: 10) so as to insert a thymidine nucleotide to the ICDH1 encoding gene (FIG. 3). This insertion has created a ‘frame shift’, which in turn lead to a premature stop codon, thus resulted in a shorter and non-functional ICDH1 protein.

Further, the inventors examined NADP-dependent ICDH activity in the ICDH1 mutated line. In general, tomato contains three NADP-dependent ICDH: ICDH1 ICDH2, and ICDH3. The inventors showed that mutating ICDH1 alone reduced most of the NADP-dependent ICDH activity throughout the ripening period (FIG. 4).

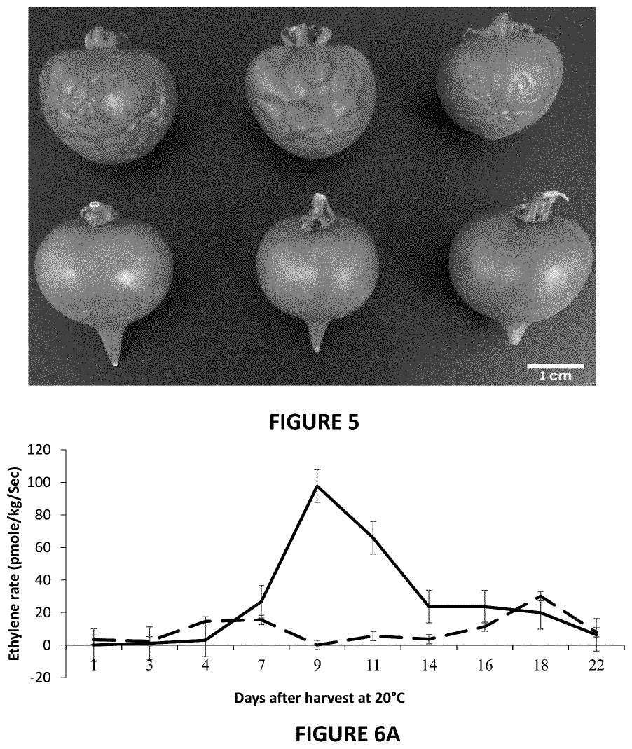

The inventors further examined the outer appearance of ICDH mutated tomatoes which were stored at room temperature for a prolonged period of time. After 50 days ICDH1 mutated tomato still had a smooth-fresh appearance, whereas the non-mutated fruit had a wrinkly-non-fresh appearance (FIG. 5).

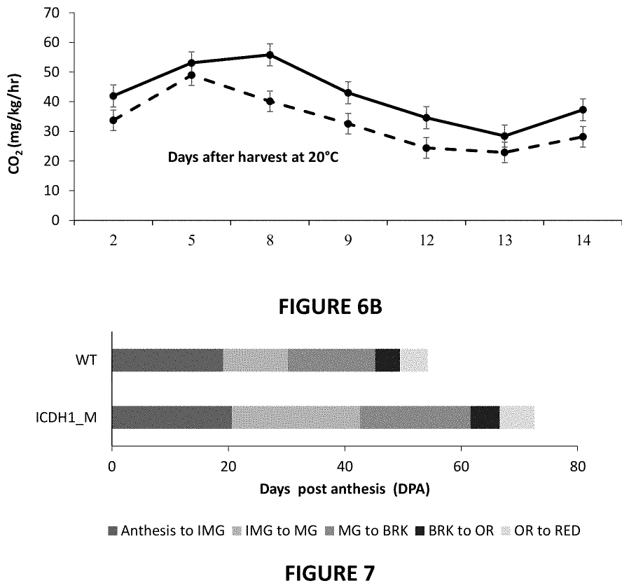

The inventors showed that in the mutated tomato (ICDH M), ethylene production was heavily reduced and a low climacteric peak appeared 18 days after harvest, compared to the non-mutated tomatoes, representing the hallmark climacteric peak of day 9 postharvest (FIG. 6A). Further, the The ripening stages were followed from athesis (full opening of the flower—the day of fertilization) till the fruit obtained the red colored. In the ICDH1 mutated tomato the duration of this process was about ten days longer.

The inventors showed that in the mutated tomato (ICDH M), ethylene production was heavily reduced and a low climacteric peak appeared 18 days after harvest, compared to the non-mutated tomatoes, representing the hallmark climacteric peak of day 9 postharvest (FIG. 6A). Further, the inventors showed that in the ICDH1 mutated tomato the duration of athesis (full opening of the flower—the day of fertilization) till the fruit obtained the red colored was about ten days longer, than in control fruits (FIG. 7).

Further, the inventors determined that silencing a cytoplasmic ICDH, namely ICDH1, resulted in fruits displaying: reduced respiration with a lower peak that appeared only on the 7th day after harvest; an ethylene peak on the 16^{th} day after harvest and overall a lower ethylene production rate throughout the experiment; and a decay of 12-20% compared to 32-56% in the control (data not shown).

Further, the inventors conducted a sequence homology analysis (e.g., multiple sequence alignment) of different species comprising ICDH1, which were found to have at least 88% identity (see Table 1 below).

TABLE 1
Homology % of ICDH1 among selected plant species.

| | | | | | | | | | |
|---|---|---|---|---|---|---|---|---|---|
| Mus | | | | | | | | | |
| acuminata | | | | | | | | | |
| Arabidopsis | 88.51 | | | | | | | | |
| thaliana | | | | | | | | | |
| Capsicum | 89.02 | 89 | | | | | | | |
| annuum | | | | | | | | | |
| Nicotiana | 89.02 | 88.51 | 95.66 | | | | | | |
| tabacum | | | | | | | | | |
| Solanum | 89.27 | 88.51 | 96.87 | 97.11 | | | | | |
| lycopersicum | | | | | | | | | |
| Solanum | 88.78 | 88.51 | 96.39 | 97.59 | 98.8 | | | | |
| tuberosum | | | | | | | | | |
| Solanum | 89.27 | 87.78 | 97.11 | 96.14 | 97.83 | 97.83 | | | |
| melongena | | | | | | | | | |
| Fragaria | 88.75 | 89.49 | 88.81 | 88.81 | 89.78 | 88.81 | 89.29 | | |
| vesca | | | | | | | | | |
| Malus | 88.29 | 89.24 | 89.08 | 89.08 | 89.81 | 89.32 | 90.05 | 92.7 | |
| domestica | | | | | | | | | |
| | Mus | Arabidopsis | Capsicum | Nicotiana | Solanum | Solanum | Solanum | Fragaria | Malus |
| | acuminata | thaliana | annuum | tabacum | lycopersicum | tuberosum | melongena | vesca | domestica |

Contrary to the above high homology levels, the inventors found that cytoplasmic and mitochondrial ICDHs and IDHs, respectively, even within the same species share very low level of sequence homology (see Table 2 below).

TABLE 2
Homology % between cytosolic (ICDHs) and mitochondrial (IDHs) isoforms in tomato
(Solanum lycopersicum).

| | | Mitochondrial isoforms | | | | Cytoplasmic isoforms | | |
|---|---|---|---|---|---|---|---|---|
| | | IDH4 | IDH3 | IDH2 | IDH1 | ICDH3 | ICDH2 | ICDH1 |
| Cytoplasmic | ICDH1 | 22.56 | 22.56 | 21.1 | 20.18 | 78.45 | 90.6 | — |
| isoforms | ICDH2 | 24.09 | 24.09 | 21.1 | 21.71 | 75.9 | — | — |
| | ICDH3 | 21.88 | 22.07 | 23.08 | 20.6 | — | — | — |
| Mitochondrial | IDH1 | 45.86 | 46.24 | 79.55 | — | — | — | — |
| isoforms | IDH2 | 47.71 | 48.41 | — | — | — | — | — |
| | IDH3 | 97.8 | — | — | — | — | — | — |
| | IDH4 | — | — | — | — | — | — | — |

Based on all of the above, the inventors suggest that the herein disclosed invention is applicable for prolonging ripening of a fruit or other plant parts, e.g., a flower, of any one of a climacteric fruit and a non-climacteric fruit by specifically targeting a cytoplasmic ICDH gene, e.g., ICDH1, for editing, such as by a gRNA and a Cas protein, so as to mutate the cytoplasmic ICDH gene, e.g., by insertion of a premature stop codon, thereby obtaining a non-functional cytoplasmic ICDH protein (or a polypeptide having a reduced and/or inferior ICDH activity compared to e.g., the wild type enzyme). The fruit, and/or other plant parts, e.g., a flower, may also harbor other beneficial traits, e.g., reduced susceptibility to pathogens, and others.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A plant comprising at least one cell comprising at least one mutation in a cytosolic isocitrate dehydrogenase (ICDH) encoding gene, wherein the plant has a reduced ethylene production rate compared to control, a reduced respiration rate compared to control, or both.

2. The plant of claim 1, comprising at least one exogenous polynucleotide comprising said at least one mutation.

3. The plant of claim 1, wherein said control is a genetic reference plant comprising the same genotype of said transgenic plant and is lacking said at least one mutation.

4. The plant of claim 1, wherein the cytosolic ICDH comprising said mutation has a reduced NADP-dependent ICDH activity compared to a control ICDH, or is inactive.

5. The plant of claim 1, wherein said at least one cytosolic ICDH encoding gene is selected from the group consisting of: ICDH1, ICDH2cyt, ICDH11, and any combination thereof.

6. The plant of claim 1, wherein said at least one cytosolic ICDH encoding gene is ICDH1.

7. A plant part derived from the plant of claim 1, optionally wherein said plant part is selected from the group consisting of: a fruit, a flower, a seed, and a leaf, optionally wherein said pant part is a fruit, optionally wherein said fruit is characterized by having a low climacteric peak on days 15-21 after harvest, optionally wherein said fruit has a development to ripening period at least 10% longer than a fruit derived from a genetic reference plant, and optionally wherein said plant part has a shelf life increased by at least 7 days compared to a plant part derived from a genetic reference plant.

8.-12. (canceled)

13. A method comprising: contacting a plant or a part derived therefrom with an effective amount of an agent, wherein said agent is capable of reducing cytosolic ICDH activity in said plant or said plant part.

14. The method of claim 13, wherein said contacting results in: reduction of ethylene production rate, reduction of respiration rate, increase of shelf life, in said plant or said part derived therefrom, or any combination thereof.

15. The method of claim 13, wherein said ICDH activity is NADP-dependent activity, optionally wherein said ICDH is selected from the group consisting of: ICDH1, ICDH2cyt and ICDH11, optionally wherein said reducing cytosolic NADP-dependent ICDH activity is constitutively or permanently reducing, optionally wherein said agent comprises at least one polynucleotide molecule, optionally wherein said at least one polynucleotide molecule encodes at least one component of the CRISPR-Cas system, optionally wherein said at least one component of the CRISPR-Cas system is a guide RNA (gRNA) or a Cas protein, optionally wherein said gRNA comprises the nucleic acid sequence 5′-CCATTCTTAAGAAATATGATGGGAGG-3′ (SEQ ID NO: 10), optionally wherein said Cas is Cas9.

16.-22. (canceled)

23. The method of claim 13, wherein said contacting is any one of: (i) pre-harvest contacting, post-harvest contacting, or both; (ii) results in said plant or said part derived therefrom being characterized by having: a low climacteric peak on days 15-21 after harvest, development to ripening period at least 10% longer than a control, a shelf life increased by at least 7 days compared to a control, a reduced decay % compared to a control, or any combination thereof, and (iii) both (i) and (ii).

24. (canceled)

25. The method of claim 13, wherein said plant part is selected form the group consisting of: a cell, a fruit, a flower, a seed, and a leaf.

26. The method of claim 13, wherein said plant part is a fruit.

27. The method of claim 13, wherein said plant is a mutated plant.

28. The method of claim 27, wherein said mutated plant comprises at least one mutation in a cytosolic ICDH encoding gene.

29. The method of claim 27, wherein said control is a genetic reference plant comprising the same genotype of said plant and lacking said at least one mutation.

30. A method for producing an agent suitable for increasing shelf life of a plant or a part derived therefrom, comprising: obtaining an agent that reduces cytosolic NADP-dependent ICDH activity, determining the ethylene production rate, respiration rate, or both in the presence of said obtained agent, and selecting at least one agent the reduces ethylene production rate, respiration rate, or both, thereby producing an agent suitable for increasing the shelf life of a plant or a part derived therefrom.

31. The method of claim 30, wherein said selected agent reduces or prevents a climacteric peak on days 7-12 after harvest.

32. The method of claim 30, further comprising a step of determining that said agent reduces or prevents a climacteric peak on days 7-12 after harvest.

33. A plant or a part derived therefrom characterized by reduced cytosolic ICDH activity obtained according to the method of claim 13.
